SEQUENCE LISTING (4 sequences)

SEQ ID NO: 1 - protein, 15 aa, Artificial
  synthetic peptide
REVQSKIGYGRQIIS

SEQ ID NO: 2 - protein, 24 aa, Artificial
  synthetic peptide
ELMTGELPYSHINNRDQIIFMVGR

SEQ ID NO: 3 - protein, 21 aa, Artificial
  synthetic peptide
TGSLPYSHIGSRDQIIFMVGR

SEQ ID NO: 4 - protein, 18 aa, Artificial
  synthetic peptide
GIVLYELMTGELPYSHIN

(19) United States
(12) Patent Application Publication
(10) Pub. No.: US 2010/0227411 A1
(43) Pub. Date: Sep. 9, 2010

(54) POLYPEPTIDE MARKERS FOR THE DIAGNOSIS OF PROSTATE CANCER

(76) Inventor: Harald Mischak, Sehnde (DE)

Correspondence Address: DARDI & HERBERT, PLLC, Moore Lake Plaza, Suite 205, 1250 East Moore Lake Drive, Fridley, MN, 55432, US

(21) Appl. No.: 12/681,529

(22) PCT Filed: Oct. 9, 2008
(86) PCT No.: PCT/EP08/63499

§ 371 Date: May 7, 2010

(30) Foreign Application Priority Data
Oct. 9, 2007 (EP) 07118113.5

(51) Int. Cl. G01N 33/68; G01N 33/50

(52) U.S. Cl. 436/86; 204/451

(57) ABSTRACT

A method for the diagnosis of prostate diseases, comprising the step of determining an amplitude or the presence or absence of at least one polypeptide marker in a sample, wherein said polypeptide marker is selected from markers 1 to 141, which are characterized by values for the molecular masses and migration times.

The present invention relates to the measuring of one or more peptide markers in a sample from a subject for the diagnosis of prostate diseases (PCA) and to a method for the diagnosis of prostate cancer, wherein the presence or absence of the peptide marker or markers is indicative of the existence of prostate cancer.

The carcinoma of the prostate gland is one of the most common cancers in males. Since complaints occur only in a stage of advanced disease, the cancer can be diagnosed in an early stage only by regular screening tests for early detection (digital rectum exam and PSA (prostate specific antigen) value in the blood). To confirm the suspicion diagnosis, a tissue specimen is withdrawn by means of fine-needle biopsy.

For the therapy, there are several possibilities that depend on the kind and stage of the tumor and on the individual needs of the patient: In an early stage, seed implantation (minimal-invasive introduction of iodine-125 radioactive emitters into the prostate) or surgical removal of the tumor and irradiation from outside are available.

At the time of diagnosis, metastasizing into other organs has already occurred in one third of the patients; at this time, the disease can hardly be healed any more. However, radiotherapy, chemotherapy or hormone therapy (combinations are possible) can delay the further spreading of the cancer. When the prostate gland is affected isolatedly, i.e., metastasizing has not occurred yet, the prognosis is favorable.

A benign tumor (adenoma) of the prostate gland, benign prostate hyperplasia (BPH), occurs much more frequently than the carcinoma. According to the prior art, it can be distinguished from a malignant carcinoma only very unreliably by means of the PSA value. In this case too, a biopsy must be effected to be able to make a clear diagnosis.

As described above, there is no non-invasive and reliable early detection of prostate cancer. To date, a clear diagnosis has been associated with invasive operations, such as biopsy. Thus, there has been a need to find a process and method for a diagnosis of prostate cancer that is as little as possible invasive, quick and inexpensive.

W0O 03/072710 describes protein biomarkers for distinguishing between prostate carcinoma cells and BPH. The application describes markers that have high uncertainties (in a range of more than ±0.5%). Due to the imprecise information, an assignment to individual markers is virtually impossible from the large number of molecules in this range. A cell lysate of prostate epithelial cells is preferably employed as a sample material, which requires a complicated sampling procedure. Due to the large number of proteins included in the definition and the difficulty of sampling, the method is little suitable.

WO 01/25791 describes methods for the diagnosis of prostate carcinomas using markers. The mentioned markers are derived from the protein semenogelin I. The stated masses are defined to a certainty of only ±0.5%. Studies show that the mentioned markers are unsuitable.

WO 2006/106129 describes polypeptide markers for the diagnosis of prostate cancer from urine samples, among others.

When this method was performed, it was found that the measurements of samples from different subjects had large variabilities, and problems arose in the evaluation.

Surprisingly, it has now been found that there are markers that offer a better significance under such circumstances, especially if they are employed in combination with an altered sample preparation.

Surprisingly, it has now been found that particular peptide markers in a sample from a subject can be used for the diagnosis of prostate cancer and for the differential diagnosis to distinguish between prostate cancer and benign prostate hyperplasia (BPH). In particular, the samples may be urine or seminal fluid samples, which are withdrawn non-invasively.

Consequently, the present invention relates to the use of the presence or absence of at least three polypeptide markers in a sample from a subject for the diagnosis of prostate diseases, wherein said polypeptide markers are selected from polypeptide marker Nos. 1 to 141 as characterized by the molecular masses and migration times as stated in Table 1.

TABLE 1
Polypeptide markers for the diagnosis of prostate
diseases and their molecular masses
and migration times (CE time in minutes):

| Number | Mass (Da) | CE time [min] |
|---|---|---|
| 1 | 858.4 | 23.3 |
| 2 | 911.5 | 25.9 |
| 3 | 1016.3 | 35.7 |
| 4 | 1016.5 | 25.8 |
| 5 | 1050.5 | 26.9 |
| 6 | 1068.6 | 21.7 |
| 7 | 1096.5 | 26.1 |
| 8 | 1128.5 | 25.7 |
| 9 | 1134.6 | 23.6 |
| 10 | 1154.6 | 25.7 |
| 11 | 1157.6 | 37.4 |
| 12 | 1179.6 | 27.1 |
| 13 | 1186.6 | 22.4 |
| 14 | 1191.6 | 36.1 |
| 15 | 1194.6 | 26.7 |
| 16 | 1200.6 | 24.2 |
| 17 | 1216.6 | 24.3 |
| 18 | 1225.6 | 26.3 |
| 19 | 1257.5 | 34.1 |
| 20 | 1265.6 | 27.1 |
| 21 | 1312.7 | 22.4 |
| 22 | 1358.4 | 36.5 |
| 23 | 1392.7 | 21.7 |
| 24 | 1449.7 | 21.8 |
| 25 | 1487.7 | 29.6 |
| 26 | 1523.7 | 22.0 |
| 27 | 1525.5 | 37.2 |
| 28 | 1552.6 | 37.2 |
| 29 | 1576.7 | 26.5 |
| 30 | 1576.7 | 46.0 |
| 31 | 1579.8 | 20.1 |
| 32 | 1584.6 | 37.7 |
| 33 | 1588.8 | 30.2 |
| 34 | 1592.8 | 22.2 |
| 35 | 1600.6 | 37.9 |
| 36 | 1627.8 | 29.5 |
| 37 | 1631.8 | 47.0 |
| 38 | 1634.9 | 29.7 |
| 39 | 1636.8 | 22.5 |
| 40 | 1640.8 | 28.1 |
| 41 | 1649.8 | 22.6 |
| 42 | 1680.8 | 30.0 |
| 43 | 1684.7 | 31.5 |
| 44 | 1687.6 | 37.8 |
| 45 | 1706.9 | 22.7 |
| 46 | 1714.6 | 37.9 |
| 47 | 1725.7 | 38.4 |
| 48 | 1728.8 | 36.8 |
| 19 | 1731.8 | 22.7 |
| 50 | 1755 | 31.4 |
| 51 | 1769.8 | 28.2 |
| 52 | 1783.9 | 39.9 |
| 53 | 1794 | 32.4 |
| 54 | 1806.9 | 23.1 |
| 55 | 1813.8 | 31.7 |
| 56 | 1819.9 | 23.4 |
| 57 | 1825.9 | 20.1 |
| 58 | 1844.6 | 34.2 |
| 59 | 1854.9 | 41.4 |
| 60 | 1860.4 | 33.6 |
| 61 | 1878.7 | 30.9 |
| 62 | 1882.9 | 20.3 |
| 63 | 1911.1 | 25.1 |
| 64 | 1925.9 | 23.3 |
| 65 | 1945.1 | 33.7 |
| 66 | 1950.9 | 35.8 |
| 67 | 1955.9 | 28.1 |
| 68 | 1969.9 | 25.3 |
| 69 | 1993 | 27.1 |
| 70 | 2031 | 32.6 |
| 71 | 2133 | 25.9 |
| 72 | 2157.1 | 22.2 |
| 73 | 2168.9 | 33.9 |
| 74 | 2184.8 | 34.2 |
| 75 | 2188 | 39.9 |
| 76 | 2210.9 | 37.7 |
| 77 | 2282.1 | 34.0 |
| 78 | 2355.2 | 22.7 |
| 19 | 2356.8 | 35.5 |
| 80 | 2414.7 | 35.6 |
| 81 | 2483.2 | 27.7 |
| 82 | 2570.3 | 42.8 |
| 83 | 2577.3 | 24.7 |
| 84 | 2599.3 | 28.0 |
| 85 | 2668.4 | 42.1 |
| 86 | 2682.2 | 22.5 |
| 87 | 2702.1 | 38.2 |
| 88 | 2726.4 | 43.2 |
| 89 | 2742.4 | 42.3 |
| 90 | 2751.5 | 29.2 |
| 91 | 2753.4 | 36.3 |
| 92 | 2754.3 | 29.7 |
| 93 | 2799.2 | 25.1 |
| 94 | 2854.5 | 34.9 |
| 95 | 2977.6 | 29.1 |
| 96 | 3001.5 | 35.5 |
| 97 | 3013.2 | 22.3 |
| 98 | 3021.5 | 23.5 |
| 99 | 3048 | 28.7 |
| 100 | 3092 | 29.7 |
| 101 | 3139.5 | 29.6 |
| 102 | 3145.6 | 38.8 |
| 103 | 3166.4 | 22.1 |
| 104 | 3168.4 | 24.7 |
| 105 | 3193.3 | 22.6 |
| 106 | 3256.6 | 33.1 |
| 107 | 3260.5 | 41.6 |
| 108 | 3292.7 | 39.5 |
| 109 | 3409.7 | 32.2 |
| 110 | 3425.7 | 31.3 |
| 111 | 3426.5 | 27.8 |
| 112 | 3530.6 | 26.2 |
| 113 | 3559.8 | 24.9 |
| 114 | 3657.8 | 40.7 |
| 115 | 3718.8 | 32.5 |
| 116 | 3734.9 | 32.5 |
| 117 | 3765.5 | 20.2 |
| 118 | 3775.7 | 25.9 |
| 119 | 3788.9 | 25.3 |
| 120 | 3858.9 | 25.8 |
| 121 | 3968.7 | 21.1 |
| 122 | 3986.8 | 20.6 |
| 123 | 3996.7 | 20.9 |
| 124 | 4043.7 | 20.4 |
| 125 | 4045 | 26.4 |
| 126 | 4098 | 24.6 |
| 127 | 4218 | 26.1 |
| 128 | 4353 | 20.2 |
| 129 | 4405 | 20.7 |
| 130 | 4410 | 20.0 |
| 131 | 4436.1 | 26.3 |
| 132 | 4672 | 23.3 |
| 133 | 4863.3 | 26.7 |
| 134 | 5000.2 | 24.4 |
| 135 | 6169.9 | 24.6 |
| 136 | 8837.7 | 21.1 |
| 137 | 8854 | 21.1 |
| 138 | 9866.8 | 20.9 |
| 139 | 10342.3 | 23.0 |
| 140 | 10753.7 | 19.7 |
| 141 | 10770.2 | 19.6 |

With the present invention, it is possible to diagnose prostate cancer at a very early stage. Thus, the disease can be cured by known methods at an early stage. The invention further enables an inexpensive, quick and reliable diagnosis with in part non-invasive or only minimal-invasive operations.

The invention further relates to differential diagnosis for distinguishing between prostate carcinoma and BPH. The differential diagnosis can be effected by using the presence or absence of at least three polypeptide markers in a sample from a subject, wherein said polypeptide markers are selected from polypeptide markers 142 to 201, which are characterized by the molecular masses and migration times as stated in Table 3. Preferably, more markers are employed.

TABLE 3
Polypeptide markers for the differential
diagnosis of prostate cancer or BPH, their
molecular masses and migration times.

| | | CE time |
|---|---|---|
| No. | Mass (Da) | (min) |
| 142 | 1210.4 | 36.5 |
| 143 | 1210.6 | 20.9 |
| 144 | 1234.6 | 27.4 |
| 145 | 1235.6 | 26.7 |
| 146 | 1268.6 | 21.4 |
| 147 | 1276.5 | 36.0 |
| 148 | 1390.5 | 37.1 |
| 149 | 1440.6 | 24.3 |
| 150 | 1467.7 | 21.7 |
| 151 | 1491.8 | 39.9 |
| 152 | 1495.6 | 37.4 |
| 153 | 1510.7 | 24.3 |
| 154 | 1523.8 | 40.5 |
| 155 | 1552.6 | 37.2 |
| 156 | 1579.8 | 29.8 |
| 157 | 1584.6 | 37.7 |
| 158 | 1624.6 | 37.7 |
| 159 | 1664.8 | 29.8 |
| 160 | 1680.8 | 30.0 |
| 161 | 1687.6 | 37.8 |
| 162 | 1706.9 | 22.7 |
| 163 | 1714.6 | 37.9 |
| 164 | 1725.7 | 38.4 |
| 165 | 1783.9 | 39.9 |
| 166 | 1794.9 | 24.0 |
| 167 | 1822.8 | 30.9 |
| 168 | 1825.9 | 20.1 |
| 169 | 1864.0 | 44.1 |
| 170 | 1882.9 | 20.3 |
| 171 | 1925.9 | 23.3 |
| 172 | 1964.0 | 31.8 |
| 173 | 2089.0 | 23.7 |
| 174 | 2118.0 | 33.0 |
| 175 | 2133.0 | 25.9 |
| 176 | 2170.0 | 33.4 |
| 177 | 2189.1 | 26.8 |
| 178 | 2282.1 | 34.0 |
| 179 | 2298.1 | 33.9 |
| 180 | 2442.2 | 34.1 |
| 181 | 2485.2 | 34.4 |
| 182 | 2599.3 | 28.0 |
| 183 | 2686.9 | 29.1 |
| 184 | 2695.3 | 23.5 |
| 185 | 2839.0 | 24.2 |
| 186 | 3092.0 | 29.7 |
| 187 | 3121.4 | 30.3 |
| 188 | 3248.5 | 30.7 |
| 189 | 3302.8 | 23.2 |
| 190 | 3333.8 | 23.8 |
| 191 | 3409.7 | 32.2 |
| 192 | 3425.7 | 31.3 |
| 193 | 3457.7 | 31.5 |
| 194 | 3478.4 | 41.8 |
| 195 | 3524.6 | 32.4 |
| 196 | 3589.8 | 25.1 |
| 197 | 3765.5 | 20.2 |
| 198 | 3839.9 | 19.7 |
| 199 | 4290.0 | 28.8 |
| 200 | 6650.9 | 25.6 |
| 201 | 10770.2 | 19.6 |

The migration time is determined by capillary electrophoresis (CE), for example, as set forth in the Example under item 2. In this Example, a glass capillary of 90 cm in length and with an inner diameter (ID) of 50 μm and an outer diameter (OD) of 360 μm is operated at an applied voltage of 30 kV. As the mobile solvent, 30% methanol, 0.5% formic acid in water is used.

It is known that the CE migration times may vary. Nevertheless, the order in which the polypeptide markers are eluted is typically the same under the stated conditions for any CE system employed. In order to balance any differences in the migration time that may nevertheless occur, the system can be normalized using standards for which the migration times are exactly known. These standards may be, for example, the polypeptides stated in the Examples (see the Example, item 3).

The characterization of the polypeptides shown in Tables 1 and 3 was determined by means of capillary electrophoresis-mass spectrometry (CE-MS), a method which has been described in detail, for example, by Neuhoff et al. (Rapid communications in mass spectrometry, 2004, Vol. 20, pages 149-156). The variation of the molecular masses between individual measurements or between different mass spectrometers is relatively small when the calibration is exact, typically within a range of ±0.03%, preferably within a range of ±0.01%.

The polypeptide markers according to the invention are proteins or peptides or degradation products of proteins or peptides. They may be chemically modified, for example, by posttranslational modifications, such as glycosylation, phosphorylation, alkylation or disulfide bridges, or by other reactions, for example, within the scope of degradation. In addition, the polypeptide markers may also be chemically altered, for example, oxidized, during the purification of the samples.

Proceeding from the parameters that determine the polypeptide markers (molecular weight and migration time), it is possible to identify the sequence of the corresponding polypeptides by methods known in the prior art.

The polypeptides according to the invention are used to diagnose prostate diseases, especially prostate cancer. “Diagnosis” means the process of knowledge gaining by assigning symptoms or phenomena to a disease or injury. In the present case, the existence of prostate cancer is concluded from the presence or absence of particular polypeptide markers. Thus, the polypeptide markers according to the invention are determined in a sample from a subject, wherein its presence or absence allows to conclude the existence of prostate cancer in the case of frequency markers. The presence or absence of a polypeptide marker can be measured by any method known in the prior art. Methods which may be used are exemplified below.

A polypeptide marker is considered present if its measured value is at least as high as its threshold value. If the measured value is lower, then the polypeptide marker is considered absent. The threshold value can be determined either by the sensitivity of the measuring method (detection limit) or defined from experience.

In the context of the present invention, the threshold value is considered to be exceeded preferably if the measured value of the sample for a certain molecular mass is at least twice as high as that of a blank sample (for example, only buffer or solvent).

The polypeptide marker or markers is/are used in such a way that its/their presence or absence is measured, wherein the presence or absence is indicative of prostate diseases, especially prostate cancer (frequency markers). Thus, there are polypeptide markers which are typically present in patients with prostate diseases, but absent or less frequent in subjects with no prostate cancer (control). In addition, there are polypeptide markers which are present in subjects with no prostate cancer, but are less frequently or not at all present in subjects with prostate cancer.

In addition or also alternatively to the determination of the presence or absence, the amplitudes may also be used for the diagnosis of prostate diseases. Amplitude markers are used in such a way that the presence or absence is not critical, but the height of the signal (the amplitude) decides if the signal is present in both groups. In Tables 2 and 4, the mean amplitudes of the corresponding signals (characterized by mass and migration time) averaged over all samples measured are stated. To achieve comparability between differently concentrated samples or different measuring methods, all peptide signals of a sample are normalized to a total amplitude of 1 million counts. Therefore, the respective mean amplitudes of the individual markers are stated as parts per million (ppm). All groups employed consist of at least 20 individual patient or control samples in order to obtain a reliable mean amplitude. The decision for a diagnosis is made as a function of how high the amplitude of the respective polypeptide markers in the patient sample is in comparison with the mean amplitudes in the control group or the “prostate group”. If the amplitude rather corresponds to the mean amplitudes of the prostate group, the existence of a prostate disease is to be considered, and if it rather corresponds to the mean amplitudes of the control group, the non-existence of a prostate disease is to be considered.

The smaller the distance between the amplitudes of the control group and the prostate group, the closer a value that lies between the two reference values has to be to one of the reference values.

One possibility is to subdivide the range between the mean amplitudes into three portions. If the value is in the lower third, this is indicative of the lower value; if the value is in the upper third, this is indicative of the upper value. If it is in the middle third, a definite statement about this marker is not possible.

TABLE 2
Amplitude markers

| | | | Pathological | | Physiological | |
|---|---|---|---|---|---|---|
| | | | prostate gland | | prostate gland | |
| | | CE time | | Mean | | Mean |
| No. | Mass (Da) | (min) | Frequency | amp. | Frequency | amp. |
| 1 | 858.4 | 23.3 | 0.56 | 45 | 0.77 | 123 |
| 2 | 911.5 | 25.9 | 0.73 | 106 | 0.86 | 260 |
| 3 | 1016.3 | 35.7 | 0.96 | 497 | 0.90 | 947 |
| 4 | 1016.5 | 25.8 | 0.95 | 385 | 0.97 | 941 |
| 5 | 1050.5 | 26.9 | 0.75 | 103 | 0.90 | 356 |
| 6 | 1068.6 | 21.7 | 0.54 | 62 | 0.78 | 225 |
| 7 | 1096.5 | 26.1 | 0.89 | 1657 | 0.96 | 3708 |
| 8 | 1128.5 | 25.7 | 0.58 | 60 | 0.80 | 158 |
| 9 | 1134.6 | 23.6 | 0.60 | 67 | 0.80 | 279 |
| 10 | 1154.6 | 25.7 | 0.57 | 95 | 0.80 | 252 |
| 11 | 1157.6 | 37.4 | 0.88 | 457 | 0.89 | 1152 |
| 12 | 1179.6 | 27.1 | 0.78 | 186 | 0.80 | 405 |
| 13 | 1186.6 | 22.4 | 0.76 | 169 | 0.82 | 386 |
| 14 | 1191.6 | 36.1 | 0.55 | 79 | 0.76 | 274 |
| 15 | 1194.6 | 26.7 | 0.94 | 924 | 0.98 | 1575 |
| 16 | 1200.6 | 24.2 | 0.99 | 2465 | 0.99 | 5193 |
| 17 | 1216.6 | 24.3 | 0.88 | 432 | 0.85 | 828 |
| 18 | 1225.6 | 26.3 | 0.87 | 155 | 0.85 | 256 |
| 19 | 1257.5 | 34.1 | 1.00 | 3161 | 0.96 | 2149 |
| 20 | 1265.6 | 27.1 | 0.86 | 2016 | 0.92 | 3539 |
| 21 | 1312.7 | 22.4 | 0.51 | 174 | 0.81 | 729 |
| 22 | 1358.4 | 36.5 | 0.99 | 1848 | 0.95 | 1405 |
| 23 | 1392.7 | 21.7 | 1.00 | 3689 | 0.97 | 2739 |
| 24 | 1449.7 | 21.8 | 1.00 | 4310 | 0.97 | 3020 |
| 25 | 1487.7 | 29.6 | 0.88 | 418 | 0.51 | 234 |
| 26 | 1523.7 | 22.0 | 0.95 | 4303 | 0.96 | 3040 |
| 27 | 1525.5 | 37.2 | 1.00 | 2319 | 0.93 | 1817 |
| 28 | 1552.6 | 37.2 | 0.99 | 2766 | 0.91 | 1862 |
| 29 | 1576.7 | 26.5 | 0.99 | 1736 | 0.93 | 921 |
| 30 | 1576.7 | 46.0 | 0.92 | 2959 | 0.57 | 1199 |
| 31 | 1579.8 | 20.1 | 0.98 | 6547 | 0.98 | 4228 |
| 32 | 1584.6 | 37.7 | 0.87 | 382 | 0.76 | 300 |
| 33 | 1588.8 | 30.2 | 0.94 | 596 | 0.46 | 114 |
| 34 | 1592.8 | 22.2 | 0.83 | 480 | 0.52 | 201 |
| 35 | 1600.6 | 37.9 | 0.88 | 388 | 0.76 | 232 |
| 36 | 1627.8 | 29.5 | 0.81 | 253 | 0.67 | 158 |
| 37 | 1631.8 | 47.0 | 0.96 | 11650 | 0.65 | 4248 |
| 38 | 1634.9 | 29.7 | 0.85 | 522 | 0.67 | 272 |
| 39 | 1636.8 | 22.5 | 1.00 | 10631 | 0.98 | 7771 |
| 40 | 1640.8 | 28.1 | 0.84 | 162 | 0.41 | 50 |
| 41 | 1649.8 | 22.6 | 0.93 | 699 | 0.82 | 422 |
| 42 | 1680.8 | 30.0 | 0.89 | 2045 | 0.86 | 1483 |
| 43 | 1684.7 | 31.5 | 0.99 | 2814 | 0.98 | 1441 |
| 44 | 1687.6 | 37.8 | 0.93 | 217 | 0.80 | 154 |
| 45 | 1706.9 | 22.7 | 1.00 | 796 | 0.93 | 408 |
| 46 | 1714.6 | 37.9 | 0.71 | 116 | 0.45 | 48 |
| 47 | 1725.7 | 38.4 | 1.00 | 2078 | 0.90 | 1407 |
| 48 | 1728.8 | 36.8 | 0.79 | 221 | 0.67 | 124 |
| 49 | 1731.8 | 22.7 | 0.88 | 172 | 0.69 | 108 |
| 50 | 1755 | 31.4 | 1.00 | 9478 | 0.92 | 3966 |
| 51 | 1769.8 | 28.2 | 0.93 | 403 | 0.69 | 137 |
| 52 | 1783.9 | 39.9 | 0.76 | 240 | 0.40 | 112 |
| 53 | 1794 | 32.4 | 0.88 | 287 | 0.78 | 202 |
| 54 | 1806.9 | 23.1 | 0.83 | 188 | 0.60 | 112 |
| 55 | 1813.8 | 31.7 | 0.99 | 2221 | 0.95 | 983 |
| 56 | 1819.9 | 23.4 | 100 | 3799 | 0.93 | 2664 |
| 57 | 1825.9 | 20.1 | 0.95 | 1494 | 0.90 | 926 |
| 58 | 1844.6 | 34.2 | 0.88 | 326 | 0.54 | 137 |
| 59 | 1854.9 | 41.4 | 0.95 | 7712 | 0.84 | 4491 |
| 60 | 1860.4 | 33.6 | 0.85 | 401 | 0.64 | 194 |
| 61 | 1878.7 | 30.9 | 0.84 | 963 | 0.69 | 534 |
| 62 | 1882.9 | 20.3 | 1.00 | 18028 | 0.98 | 12004 |
| 63 | 1911.1 | 25.1 | 0.99 | 62532 | 0.96 | 39024 |
| 64 | 1925.9 | 23.3 | 0.75 | 120 | 0.28 | 44 |
| 65 | 1945.1 | 33.7 | 0.86 | 197 | 0.74 | 180 |
| 66 | 1950.9 | 35.8 | 0.97 | 593 | 0.77 | 357 |
| 67 | 1955.9 | 28.1 | 0.82 | 345 | 0.44 | 86 |
| 68 | 1969.9 | 25.3 | 0.89 | 915 | 0.61 | 242 |
| 69 | 1993 | 27.1 | 0.80 | 104 | 0.34 | 33 |
| 70 | 2031 | 32.6 | 0.92 | 838 | 0.80 | 538 |
| 71 | 2133 | 25.9 | 0.98 | 714 | 0.70 | 278 |
| 72 | 2157.1 | 22.2 | 0.79 | 347 | 0.90 | 736 |
| 73 | 2168.9 | 33.9 | 0.99 | 706 | 0.91 | 493 |
| 74 | 2184.8 | 34.2 | 0.95 | 610 | 0.88 | 300 |
| 75 | 2188 | 39.9 | 0.97 | 1452 | 0.81 | 606 |
| 76 | 2210.9 | 37.7 | 0.84 | 274 | 0.64 | 183 |
| 77 | 2282.1 | 34.0 | 0.99 | 5409 | 0.99 | 5449 |
| 78 | 2355.2 | 22.7 | 0.99 | 554 | 0.97 | 956 |
| 79 | 2356.8 | 35.5 | 0.98 | 571 | 0.75 | 230 |
| 80 | 2414.7 | 35.6 | 0.78 | 471 | 0.61 | 164 |
| 81 | 2483.2 | 27.7 | 0.76 | 155 | 0.84 | 353 |
| 82 | 2570.3 | 42.8 | 0.99 | 5993 | 0.80 | 3366 |
| 83 | 2577.3 | 24.7 | 0.96 | 297 | 0.82 | 516 |
| 84 | 2599.3 | 28.0 | 0.95 | 1856 | 0.79 | 180 |
| 85 | 2668.4 | 42.1 | 0.90 | 529 | 0.72 | 333 |
| 86 | 2682.2 | 22.5 | 0.99 | 844 | 0.91 | 588 |
| 87 | 2702.1 | 38.2 | 0.82 | 332 | 0.64 | 137 |
| 88 | 2726.4 | 43.2 | 0.99 | 4394 | 0.80 | 2280 |
| 89 | 2742.4 | 42.3 | 0.87 | 592 | 0.50 | 188 |
| 90 | 2751.5 | 29.2 | 0.87 | 239 | 0.55 | 139 |
| 91 | 2753.4 | 36.3 | 0.86 | 150 | 0.64 | 79 |
| 92 | 2754.3 | 29.7 | 0.88 | 318 | 0.77 | 182 |
| 93 | 2799.2 | 25.1 | 1.00 | 3236 | 1.00 | 2169 |
| 94 | 2854.5 | 34.9 | 0.99 | 3786 | 0.97 | 2215 |
| 95 | 2977.6 | 29.1 | 0.84 | 179 | 0.47 | 66 |
| 96 | 3001.5 | 35.5 | 0.99 | 12587 | 0.98 | 7515 |
| 97 | 3013.2 | 22.3 | 0.91 | 1824 | 0.98 | 3609 |
| 98 | 3021.5 | 23.5 | 1.00 | 1642 | 0.85 | 1055 |
| 99 | 3048 | 28.7 | 0.82 | 230 | 0.72 | 112 |
| 100 | 3092 | 29.7 | 0.99 | 780 | 0.89 | 618 |
| 101 | 3139.5 | 29.6 | 0.97 | 1549 | 0.86 | 673 |
| 102 | 3145.6 | 38.8 | 0.97 | 1147 | 0.84 | 663 |
| 103 | 3166.4 | 22.1 | 0.82 | 409 | 0.51 | 159 |
| 104 | 3168.4 | 24.7 | 0.82 | 211 | 0.91 | 477 |
| 105 | 3193.3 | 22.6 | 0.81 | 383 | 0.88 | 783 |
| 106 | 3256.6 | 33.1 | 0.90 | 1008 | 0.71 | 564 |
| 107 | 3260.5 | 41.6 | 0.86 | 371 | 0.55 | 205 |
| 108 | 3292.7 | 39.5 | 0.99 | 5967 | 0.91 | 3920 |
| 109 | 3409.7 | 32.2 | 0.89 | 386 | 0.84 | 287 |
| 110 | 3425.7 | 31.3 | 1.00 | 1098 | 0.97 | 1200 |
| 111 | 3426.5 | 27.8 | 0.81 | 136 | 0.45 | 45 |
| 112 | 3530.6 | 26.2 | 0.95 | 775 | 0.73 | 317 |
| 113 | 3559.8 | 24.9 | 0.96 | 896 | 0.80 | 633 |
| 114 | 3657.8 | 40.7 | 0.96 | 964 | 0.80 | 648 |
| 115 | 3718.8 | 32.5 | 0.97 | 364 | 0.95 | 695 |
| 116 | 3734.9 | 32.5 | 0.91 | 200 | 0.93 | 410 |
| 117 | 3765.5 | 20.2 | 0.60 | 199 | 0.26 | 40 |
| 118 | 3775.7 | 25.9 | 0.99 | 1537 | 0.88 | 1110 |
| 119 | 3788.9 | 25.3 | 0.78 | 258 | 0.57 | 144 |
| 120 | 3858.9 | 25.8 | 0.97 | 975 | 0.82 | 690 |
| 121 | 3968.7 | 21.1 | 0.95 | 1430 | 0.83 | 904 |
| 122 | 3986.8 | 20.6 | 0.99 | 4017 | 0.86 | 1722 |
| 123 | 3996.7 | 20.9 | 0.83 | 707 | 0.39 | 197 |
| 124 | 4043.7 | 20.4 | 0.92 | 1038 | 0.67 | 496 |
| 125 | 4045 | 26.4 | 0.98 | 2449 | 0.88 | 1561 |
| 126 | 4098 | 24.6 | 0.94 | 370 | 0.90 | 780 |
| 127 | 4218 | 26.1 | 0.97 | 4520 | 0.88 | 2787 |
| 128 | 4353 | 20.2 | 0.98 | 4815 | 0.88 | 2186 |
| 129 | 4405 | 20.7 | 0.86 | 704 | 0.59 | 244 |
| 130 | 4410 | 20.0 | 0.82 | 997 | 0.60 | 472 |
| 131 | 4436.1 | 26.3 | 0.95 | 2663 | 0.87 | 1538 |
| 132 | 4672 | 23.3 | 0.90 | 309 | 0.77 | 196 |
| 133 | 4863.3 | 26.7 | 0.61 | 158 | 0.76 | 389 |
| 134 | 5000.2 | 24.4 | 0.99 | 2538 | 0.98 | 1743 |
| 135 | 6169.9 | 24.6 | 0.78 | 444 | 0.46 | 282 |
| 136 | 8837.7 | 21.1 | 0.94 | 1669 | 0.71 | 1430 |
| 137 | 8854 | 21.1 | 0.63 | 441 | 0.86 | 1383 |
| 138 | 9866.8 | 20.9 | 0.99 | 1874 | 0.83 | 944 |
| 139 | 10342.3 | 23.0 | 0.77 | 1771 | 0.35 | 279 |
| 140 | 10753.7 | 19.7 | 0.76 | 4585 | 0.33 | 247 |
| 141 | 10770.2 | 19.6 | 0.52 | 2190 | 0.14 | 59 |

For the differential diagnosis between PCA and BPH, Table 4 shows polypeptide markers that are typically present in patients with prostate carcinoma, such as the marker No. 145, but are not or rarely present in subjects with BPH. Further, there are polypeptide markers that are present in subjects with BPH, but occur less frequently or not at all in subjects with PCA, for example, the polypeptide marker No. 163.

TABLE 4

| | | | PCa | | BPH | |
|---|---|---|---|---|---|---|
| | Mass | CE time | | Mean | Frequency | Mean |
| No. | (Da) | (min) | Frequency (%) | amp. | (%) | amp. |
| 142 | 1210.4 | 36.5 | 0.50 | 149 | 0.61 | 243 |
| 143 | 1210.6 | 20.9 | 0.65 | 121 | 0.54 | 101 |
| 144 | 1234.6 | 27.4 | 0.96 | 841 | 0.98 | 977 |
| 145 | 1235.6 | 26.7 | 0.69 | 550 | 0.55 | 458 |
| 146 | 1268.6 | 21.4 | 0.37 | 52 | 0.46 | 86 |
| 147 | 1276.5 | 36.0 | 0.98 | 2879 | 0.99 | 3241 |
| 148 | 1390.5 | 37.1 | 1.00 | 19627 | 0.97 | 21822 |
| 149 | 1440.6 | 24.3 | 0.66 | 135 | 0.62 | 107 |
| 150 | 1467.7 | 21.7 | 0.41 | 89 | 0.54 | 102 |
| 151 | 1491.8 | 39.9 | 0.68 | 217 | 0.76 | 269 |
| 152 | 1495.6 | 37.4 | 0.68 | 134 | 0.79 | 162 |
| 153 | 1510.7 | 24.3 | 0.39 | 47 | 0.50 | 77 |
| 154 | 1523.8 | 40.5 | 0.98 | 7023 | 0.98 | 7856 |
| 155 | 1552.6 | 37.2 | 0.98 | 2130 | 0.99 | 2530 |
| 156 | 1579.8 | 29.8 | 0.98 | 1824 | 0.99 | 2061 |
| 157 | 1584.6 | 37.7 | 0.82 | 301 | 0.89 | 397 |
| 158 | 1624.6 | 37.7 | 0.99 | 1020 | 0.99 | 1159 |
| 159 | 1664.8 | 29.8 | 0.92 | 386 | 0.94 | 475 |
| 160 | 1680.8 | 30.0 | 0.82 | 1626 | 0.92 | 2182 |
| 161 | 1687.6 | 37.8 | 0.88 | 161 | 0.88 | 193 |
| 162 | 1706.9 | 22.7 | 0.99 | 705 | 0.97 | 624 |
| 163 | 1714.6 | 37.9 | 0.52 | 72 | 0.67 | 105 |
| 164 | 1725.7 | 38.4 | 1.00 | 1646 | 0.99 | 1964 |
| 165 | 1783.9 | 39.9 | 0.63 | 175 | 0.73 | 224 |
| 166 | 1794.9 | 24.0 | 0.98 | 1179 | 0.96 | 1305 |
| 167 | 1822.8 | 30.9 | 0.67 | 192 | 0.59 | 148 |
| 168 | 1825.9 | 20.1 | 0.95 | 1629 | 0.96 | 1370 |
| 169 | 1864.0 | 44.1 | 0.70 | 874 | 0.77 | 1182 |
| 170 | 1882.9 | 20.3 | 0.99 | 18958 | 0.99 | 17650 |
| 171 | 1925.9 | 23.3 | 0.74 | 121 | 0.62 | 105 |
| 172 | 1964.0 | 31.8 | 0.54 | 96 | 0.42 | 71 |
| 173 | 2089.0 | 23.7 | 0.56 | 76 | 0.46 | 52 |
| 174 | 2118.0 | 33.0 | 0.43 | 42 | 0.55 | 61 |
| 175 | 2133.0 | 25.9 | 0.98 | 711 | 0.94 | 589 |
| 176 | 2170.0 | 33.4 | 0.34 | 201 | 0.48 | 295 |
| 177 | 2189.1 | 26.8 | 0.94 | 889 | 0.92 | 640 |
| 178 | 2282.1 | 34.0 | 1.00 | 5015 | 0.99 | 5720 |
| 179 | 2298.1 | 33.9 | 0.49 | 95 | 0.58 | 127 |
| 180 | 2442.2 | 34.1 | 0.97 | 553 | 0.98 | 708 |
| 181 | 2485.2 | 34.4 | 0.49 | 42 | 0.57 | 67 |
| 182 | 2599.3 | 28.0 | 0.94 | 1303 | 0.93 | 1675 |
| 183 | 2686.9 | 29.1 | 0.54 | 102 | 0.66 | 117 |
| 184 | 2695.3 | 23.5 | 0.99 | 2295 | 1.00 | 2058 |
| 185 | 2839.0 | 24.2 | 0.77 | 589 | 0.86 | 687 |
| 186 | 3092.0 | 29.7 | 0.95 | 704 | 0.99 | 854 |
| 187 | 3121.4 | 30.3 | 0.93 | 585 | 0.94 | 690 |
| 188 | 3248.5 | 30.7 | 1.00 | 1184 | 1.00 | 1363 |
| 189 | 3302.8 | 23.2 | 0.65 | 102 | 0.68 | 135 |
| 190 | 3333.8 | 23.8 | 0.41 | 128 | 0.32 | 77 |
| 191 | 3409.7 | 32.2 | 0.81 | 357 | 0.92 | 508 |
| 192 | 3425.7 | 31.3 | 1.00 | 1132 | 1.00 | 1339 |
| 193 | 3457.7 | 31.5 | 1.00 | 13651 | 1.00 | 15121 |
| 194 | 3478.4 | 41.8 | 0.53 | 315 | 0.64 | 368 |
| 195 | 3524.6 | 32.4 | 0.49 | 285 | 0.61 | 397 |
| 196 | 3589.8 | 25.1 | 0.93 | 778 | 0.91 | 699 |
| 197 | 3765.5 | 20.2 | 0.64 | 200 | 0.43 | 132 |
| 198 | 3839.9 | 19.7 | 0.92 | 1868 | 0.92 | 1621 |
| 199 | 4290.0 | 28.8 | 0.97 | 2383 | 0.99 | 2698 |
| 200 | 6650.9 | 25.6 | 0.69 | 161 | 0.54 | 134 |
| 201 | 10770.2 | 19.6 | 0.44 | 1814 | 0.60 | 2746 |

The subject from which the sample in which the presence or absence or the amplitude of one or more polypeptide markers is determined is derived may be any subject which is capable of suffering from prostate diseases, for example, an animal or human. Preferably, the subject is a mammal, such as a dog or a horse, and most preferably, it is a human.

Preferably, for the application of the invention, not just one polypeptide marker, but a combination of markers are used to diagnose prostate cancer, wherein the existence of prostate diseases is concluded from their presence or absence and/or the height of the amplitude. By comparing a plurality of polypeptide markers, a bias in the overall result from a few individual deviations from the typical presence probability in the sick or control individual can be reduced or avoided.

The sample in which the presence or absence or amplitude of the polypeptide marker or markers according to the invention is measured may be any sample which is obtained from the body of the subject. The sample is a sample which has a polypeptide composition suitable for providing information about the state of the subject (prostate cancer or not). For example, it may be urine, sperm, seminal fluid (sperm without spermatozoa). Preferably, it is a liquid sample.

In a preferred embodiment, the sample is a urine sample.

Urine samples can be taken as known in the prior art. Preferably, a first stream or midstream urine sample is used in the context of the present invention. For example, the urine sample may be taken by means of a catheter or also by means of an urination apparatus as described in WO 01/74275.

The presence or absence of a polypeptide marker in the sample may be determined by any method known in the prior art that is suitable for measuring polypeptide markers. Such methods are known to the skilled person. In principle, the presence or absence of a polypeptide marker can be determined by direct methods, such as mass spectrometry, or indirect methods, for example, by means of ligands.

If required or desirable, the sample from the subject, for example, the urine sample, may be pretreated by any suitable means and, for example, purified or separated before the presence or absence of the polypeptide marker or markers is measured. The treatment may comprise, for example, purification, separation, dilution or concentration. The methods may be, for example, centrifugation, filtration, ultrafiltration, dialysis, precipitation or chromatographic methods, such as affinity separation or separation by means of ion-exchange chromatography, or electrophoretic separation. Particular examples thereof are gel electrophoresis, two-dimensional polyacrylamide gel electrophoresis (2D-PAGE), capillary electrophoresis, metal affinity chromatography, immobilized metal affinity chromatography (IMAC), lectin-based affinity chromatography, liquid chromatography, high-performance liquid chromatography (HPLC), normal and reverse-phase HPLC, cation-exchange chromatography and selective binding to surfaces. All these methods are well known to the skilled person, and the skilled person will be able to select the method as a function of the sample employed and the method for determining the presence or absence of the polypeptide marker or markers.

In one embodiment of the invention, the sample, before being measured, is separated by capillary electrophoresis, purified by ultracentrifugation and/or divided by ultrafiltration into fractions which contain polypeptide markers of a particular molecular size.

Preferably, a mass-spectrometric method is used to determine the presence or absence of a polypeptide marker, wherein a purification or separation of the sample may be performed upstream from such method. As compared to the currently employed methods, mass-spectrometric analysis has the advantage that the concentration of many (>100) polypeptides of a sample can be determined by a single analysis. Any type of mass spectrometer may be employed. By means of mass spectrometry, it is possible to measure 10 fmol of a polypeptide marker, i.e., 0.1 ng of a 10 kDa protein, as a matter of routine with a measuring accuracy of about ±0.01% in a complex mixture. In mass spectrometers, an ion-forming unit is coupled with a suitable analytic device. For example, electrospray-ionization (ESI) interfaces are mostly used to measure ions in liquid samples, whereas the matrix-assisted laser desorption/ionization (MALDI) technique is used for measuring ions from a sample crystallized with a matrix. For analyzing the ions formed, quadrupoles, ion traps or time-of-flight (TOF) analyzers may be used.

In electrospray ionization (ESI), the molecules present in solution are atomized, inter alia, under the influence of high voltage (e.g., 1-8 kV), which forms charged droplets that become smaller from the evaporation of the solvent. Finally, so-called Coulomb explosions cause the formation of free ions, which can then be analyzed and detected.

In the analysis of the ions by means of TOF, a particular acceleration voltage is applied which confers an equal amount of kinetic energy to the ions. Thereafter, the time that the respective ions take to travel a particular drifting distance through the flying tube is measured very accurately.

Since with equal amounts of kinetic energy, the velocity of the ions depends on their mass, the latter can thus be determined. TOF analyzers have a very high scanning speed and therefore reach a very high resolution.

Preferred methods for the determination of the presence and absence of polypeptide markers include gas-phase ion spectrometry, such as laser desorption/ionization mass spectrometry, MALDI-TOF MS, SELDI-TOF MS (surface-enhanced laser desorption/ionization), LC-MS (liquid chromatography/mass spectrometry), 2D-PAGE/MS and capillary electrophoresis-mass spectrometry (CE-MS). All methods mentioned are known to the skilled person.

A particularly preferred method is CE-MS, in which capillary electrophoresis is coupled with mass spectrometry. This method has been described in some detail, for example, in the German Patent Application DE 10021737, in Kaiser et al. (J Chromatogr A, 2003, Vol. 1013: 157-171, and Electrophoresis, 2004, 25: 2044-2055) and in Wittke et al. (Journal of Chromatography A, 2003, 1013: 173-181). The CE-MS technology allows to determine the presence of some hundreds of polypeptide markers of a sample simultaneously within a short time and in a small volume with high sensitivity. After a sample has been measured, a pattern of the measured polypeptide markers is prepared. This pattern can be compared with reference patterns of sick or healthy subjects. In most cases, it is sufficient to use a limited number of polypeptide markers for the diagnosis of prostate cancer and the differential diagnosis between prostate cancer and BPH. A CE-MS method which includes CE coupled on-line to an ESI-TOF MS device is further preferred.

For CE-MS, the use of volatile solvents is preferred, and it is best to work under essentially salt-free conditions. Examples of suitable solvents include acetonitrile, methanol and the like. The solvents can be diluted with water or admixed with a weak acid (e.g., from 0.1% to 1% formic acid) in order to protonate the analyte, preferably the polypeptides.

By means of capillary electrophoresis, it is possible to separate molecules by their charge and size. Neutral particles will migrate at the speed of the electro-osmotic flow upon application of a current, while cations are accelerated towards the cathode, and anions are delayed. The advantage of capillaries in electro-phoresis resides in their favorable ratio of surface to volume, which enables a good dissipation of the Joule heat generated during the current flow. This in turn allows high voltages (usually up to 30 kV) to be applied and thus a high separating performance and short times of analysis.

In capillary electrophoresis, silica glass capillaries having inner diameters of typically from 50 to 75 μm are usually employed. The lengths employed are from 30 to 100 cm. In addition, the capillaries are usually made of plastic-coated silica glass. The capillaries may be both untreated, i.e., expose their hydrophilic groups on the interior surface, or coated on the interior surface. A hydrophobic coating may be used to improve the resolution. In addition to the voltage, a pressure may also be applied, which typically is within a range of from 0 to 1 psi. The pressure may also be applied only during the performance or altered meanwhile.

In a preferred method for measuring polypeptide markers, the markers of the sample are separated by means of capillary electrophoresis, then directly ionized and transferred on-line to a mass spectrometer coupled thereto for detection.

In the method according to the invention, it is advantageous to use several polypeptide markers for the diagnosis of prostate cancer. In particular, at least three polypeptide markers may be used, for example, markers 1, 2 and 3; 1, 2 and 4; etc.

More preferred is the use of at least 4, 5 or 6 markers.

Even more preferred is the use of at least 11 markers, for example, markers 1 to 11.

Preferably, a subgroup of the markers are measured, wherein the markers are selected in such a way as to be found in the sample with a high probability. Depending on the frequencies of the markers, they are selected in such a way that at least half the markers are found in a sample with 90% probability. For example, if only 2 markers are analyzed, for example, markers 1 and 2 in a patient with a pathological prostate gland, then the probability that none of the markers is found in the sample is 12%, i.e., at least 1 marker is found with 88% probability. Therefore, it is more advantageous to use markers 2 and 3. In this case, the probability that no marker is found is only about 1%, i.e., the probability that at least one of the markers is found is almost 99%. It is known to the skilled person how to use statistical methods to select suitable marker combinations, wherein preferably at least 6, more preferably at least 10, even more preferably at least 20 markers are analyzed.

Most preferred is the use of all markers listed in Tables 1 or 3.

Several markers may also be used for the differential diagnosis between PCA and BPH. In particular, at least three polypeptide markers can be used.

More preferred is the use of at least 4, 5 or 6 markers. Even more preferred is the use of at least 7 markers.

Most preferred is the use of all the markers listed in Tables 2 or 4.

In order to determine the probability of the existence of prostate cancer when several markers are used, statistic methods known to the skilled person may be used. For example, the Random Forests method described by Weissinger et al. (Kidney Int., 2004, 65: 2426-2434) may be used by using a computer program such as S-Plus.

In the natural ageing process of humans, the glomerular filtration performance of the kidney decreases (J. Gerontol. 31 (1976) 155), which results in a higher abundance of larger proteins in the urine. This process, which may exhibit a large variability among individuals in terms of its intensity, complicates the comparability of measurements for the determination of pathological changes of the prostate gland from urine. For this reason, the sample preparation of urine samples had to be improved to remove these disturbing molecules. The improved protocol removes all molecules larger than 20 kDa, which results in a greatly improved mutual comparability of the urine samples.

### EXAMPLE

### 1. Sample Preparation

For the subsequent CE-MS measurement, the proteins which are also contained in urine in a higher concentration, such as albumin and immunoglobulins, had to be separated off by ultrafiltration. Thus, 700 μl of urine was removed and admixed with 700 μl of filtration buffer (2 M urea, 10 mM ammonia, 0.02% SDS). This 1.4 ml of sample volume was ultrafiltrated (20 kDa, Sartorius, Göttingen, DE). The ultrafiltration was performed at 3000 rpm in a centrifuge until 1.1 ml of ultrafiltrate was obtained. The 1.1 ml of filtrate obtained was then applied to a PD 10 column (Amersham Bioscience, Uppsala, Sweden), and desalted against 2.5 ml of 0.01% NH₄OH and lyophilized. For the CE-MS measurement, the polypeptides were then resuspended with 20 μl of water (HPLC grade, Merck).

### 2. CE-MS Measurement

The CE-MS measurements were performed with a capillary electrophoresis system from Beckman Coulter (P/ACE MDQ System; Beckman Coulter Inc., Fullerton, USA) and an ESI-TOF mass spectrometer from Bruker (micro-TOF MS, Bruker Daltonik, Bremen, Germany).

The CE capillaries were supplied by Beckman Coulter and had an ID/OD of 50/360 μm and a length of 90 cm. The mobile phase for the CE separation consisted of 20% acetonitrile and 0.25% formic acid in water. For the “sheath flow” on the MS, 30% isopropanol with 0.5% formic acid was used at a flow rate of 2 μl/min. The coupling of CE and MS was realized by a CE-ESI-MS Sprayer Kit (Agilent Technologies, Waldbronn, Germany).

For injecting the sample, a pressure of from 1 to a maximum of 6 psi was applied, and the duration of the injection was 99 seconds. With these parameters, about 150 nl of the sample was injected into the capillary, which corresponds to about 10% of the capillary volume. A stacking technique was used to concentrate the sample in the capillary. Thus, before the sample was injected, a 1 M NH₃ solution was injected for 7 seconds (at 1 psi), and after the sample was injected, a 2 M formic acid solution was injected for 5 seconds. After the separation voltage (30 kV) was applied, the analytes were automatically concentrated between these solutions.

The subsequent CE separation was performed with a pressure method: 40 minutes at 0 psi, then 0.1 psi for 2 min, 0.2 psi for 2 min, 0.3 psi for 2 min, 0.4 psi for 2 min, and finally 0.5 psi for 32 min. The total duration of a separation run was thus 80 minutes.

In order to obtain as good as possible a signal intensity on the side of the MS, the nebulizer gas was set to the lowest possible value. The voltage applied to the spray needle for generating the electrospray was 3700-4100 V. The remaining settings at the mass spectrometer were optimized for peptide detection according to the manufacturer's protocol. The spectra were recorded over a mass range of m/z 400 to m/z 3000 and accumulated every 3 seconds.

In addition to the modified sample preparation, the comparability of the individual urine samples was improved by calibrating the masses and migration times. For calibration, a “local linear regression” with reference polypeptides as known to the skilled person was performed. Any polypeptides defined by an exact mass and migration time can be used as reference proteins, and polypeptides having a high signal-to-noise ratio are preferably used, “house keeping proteins” being more preferably used. “House keeping proteins” are protein standards that are naturally present in the majority of the urine samples in slightly varying amounts.

### 3. Standards for the CE Measurement

For checking the CE measurement and calibrating, the following proteins or polypeptides which are characterized by the stated CE migration times under the selected conditions were employed:

| Protein/polypeptide | Migration time |
|---|---|
| Aprotinin (SIGMA, Taufkirchen, DE, Cat. # A1153) | 19.3 min |
| Ribonuclease (SIGMA, Taufkirchen, DE, Cat. # R4875) | 19.55 min |
| Lysozyme (SIGMA, Taufkirchen, DE, Cat. # L7651) | 19.28 min |
| “REV”, Sequence: REVQSKIGYGRQIIS | 20.95 min |
| “ELM”, Sequence: ELMTGELPYSHINNRDQIIFMVGR | 23.49 min |
| “KINCON”, Sequence: TGSLPYSHIGSRDQIIFMVGR | 22.62 min |
| “GIVLY” Sequence: GIVLYELMTGELPYSHIN | 32.2 min |

The proteins/polypeptides were employed at a concentration of 10 pmol/μl each in water. “REV”, “ELM, “KINCON” and “GIVLY” are synthetic peptides.

The above described modifications resulted in an improvement over WO 03/072710 A2 in view of both the detection limits and the reproducible detectability of polypeptides. Thus, polypeptides could be identified that indicate pathological alterations of the prostate gland (Pea (prostate cancer), high grade PIN (prostatic intra-epithelial neoplasia), BPH (benign prostate hyperplasia) and ASAP (atypical small acinar proliferation)) as compared to a healthy, physiological prostate gland (Table 1). In addition, due to these improvements, polypeptides could be identified that allow a distinction between PCa and BPH that is improved as compared to WO 03/072710 A2 (Table 2).

The molecular masses of the peptides and the m/z ratios of the individual charge states visible in MS are listed in the following Table:

| | H | | | | | | |
|---|---|---|---|---|---|---|---|
| | (mono) | | | | | | |
| | 1.0079 | 1.0079 | 1.0079 | 1.0079 | 1.0079 | 1.0079 | 1.0079 |
| | m/z | | | | | | |
| | Aprotinin | Ribonuclease | Lysozyme | REV | KINCON | ELM | GIVLY |
| | Mono | Mono | Mono | Mono | Mono | Mono | Mono |
| | Mass | Mass | Mass | Mass | Mass | Mass | Mass |
| 0 | 6513.09 | 13681.32 | 14303.88 | 1732.96 | 2333.19 | 2832.41 | 2048.03 |
| 1 | 6514.0979 | 13682.328 | 14304.888 | 1733.9679 | 2334.1979 | 2833.4179 | 2049.0379 |
| 2 | 3257.5529 | 6841.6679 | 7152.9479 | 867.4879 | 1167.6029 | 1417.2129 | 1025.0229 |
| 3 | 2172.0379 | 4561.4479 | 4768.9679 | 578.6612 | 778.7379 | 945.1446 | 683.6846 |
| 4 | 1629.2804 | 3421.3379 | 3576.9779 | 434.2479 | 584.3054 | 709.1104 | 513.0154 |
| 5 | 1303.6259 | 2737.2719 | 2861.7839 | 347.5999 | 467.6459 | 567.4899 | 410.6139 |
| 6 | 1086.5229 | 2281.2279 | 2384.9879 | 289.8346 | 389.8729 | 473.0762 | 342.3462 |
| 7 | 931.4494 | 1955.4822 | 2044.4193 | 248.5736 | 334.3208 | 405.6379 | 293.5836 |
| 8 | 815.1442 | 1711.1729 | 1788.9929 | 217.6279 | 292.6567 | 355.0592 | 257.0117 |
| 9 | 724.6846 | 1521.1546 | 1590.3279 | 193.559 | 260.2512 | 315.7201 | 228.5668 |
| 10 | 652.3169 | 1369.1399 | 1431.3959 | 174.3039 | 234.3269 | 284.2489 | 205.8109 |
| 11 | 593.107 | 1244.7643 | 1301.3606 | 158.5497 | 213.1161 | 258.4997 | 187.1924 |
| 12 | 543.7654 | 1141.1179 | 1192.9979 | 145.4212 | 195.4404 | 237.0421 | 171.6771 |
| 13 | 502.0148 | 1053.4171 | 1101.3063 | 134.3125 | 180.4841 | 218.8856 | 158.5486 |

### Performance of the Markers Described in WO 01/25791

WO 01/25791 mentions eleven markers (see claim 2 thereof), and the accuracy of mass determination is stated to be 0.5%. If it is considered that these are only fragments of semenogelin I, the following numbers of possible fragments of semenogelin are obtained:

| | Number of possible | |
|---|---|---|
| Molecular masses of the biomarker | fragments from seminogelin I | |
| polypeptides for prostate carcinoma | Mass | Mass |
| [g/mol] | deviation 0.5% | deviation 0.03% |
| 2776 | 100 | 8 |
| 4423 | 182 | 16 |
| 4480 | 173 | 8 |
| 5753 | 212 | 11 |
| 6098 | 214 | 7 |
| 6270 | 217 | 19 |
| 6998 | 248 | 12 |
| 7843 | 270 | 11 |
| 8030 | 265 | 23 |
| 8240 | 281 | 12 |
| 8714 | 299 | 23 |

Further, WO 01/25791 discloses markers that speak against the existence of a prostate carcinoma. The number of possible fragments is contained in the following Table:

| | Number of possible | |
|---|---|---|
| Molecular masses of the | fragments from seminogelin I | |
| biomarker polypeptides for | | Mass |
| prostate carcinoma [g/mol] | Mass deviation 0.5% | deviation 0.03% |
| 2095 | 83 | 2 |
| 2276 | 84 | 5 |
| 2530 | 99 | 6 |
| 3030 | 106 | 4 |
| 3038 | 116 | 10 |
| 3224 | 127 | 7 |
| 3600 | 139 | 6 |
| 3835 | 146 | 9 |
| 3915 | 150 | 16 |
| 3933 | 153 | 23 |
| 4175 | 161 | 14 |

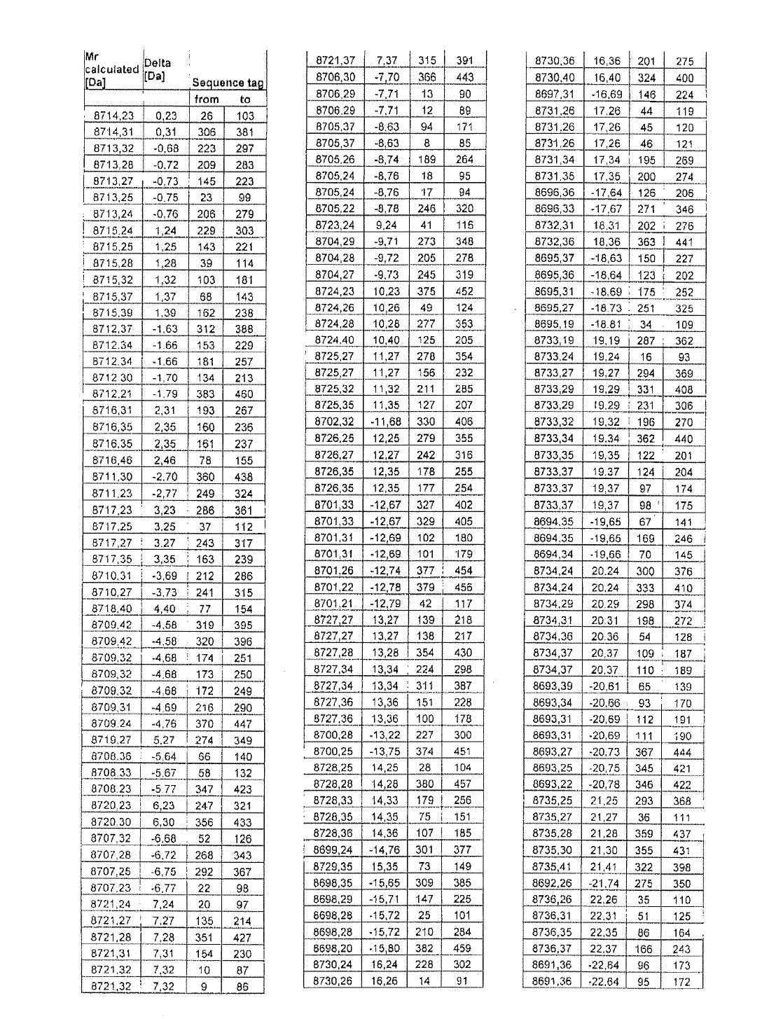

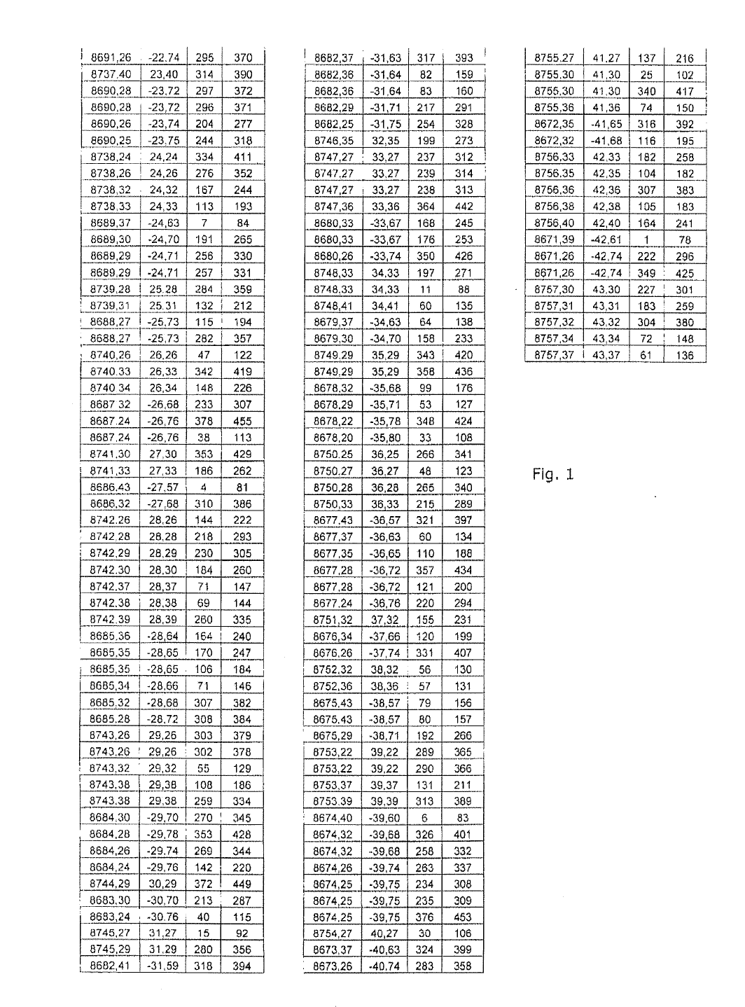

Illustratively for one mass, FIG. 1 shows the large number of sequences that are possible in this weight range.

In the next step, it was tried to analyze the corresponding proteins in urine samples from patients with prostate carcinoma or BPH. Only six markers could be found.

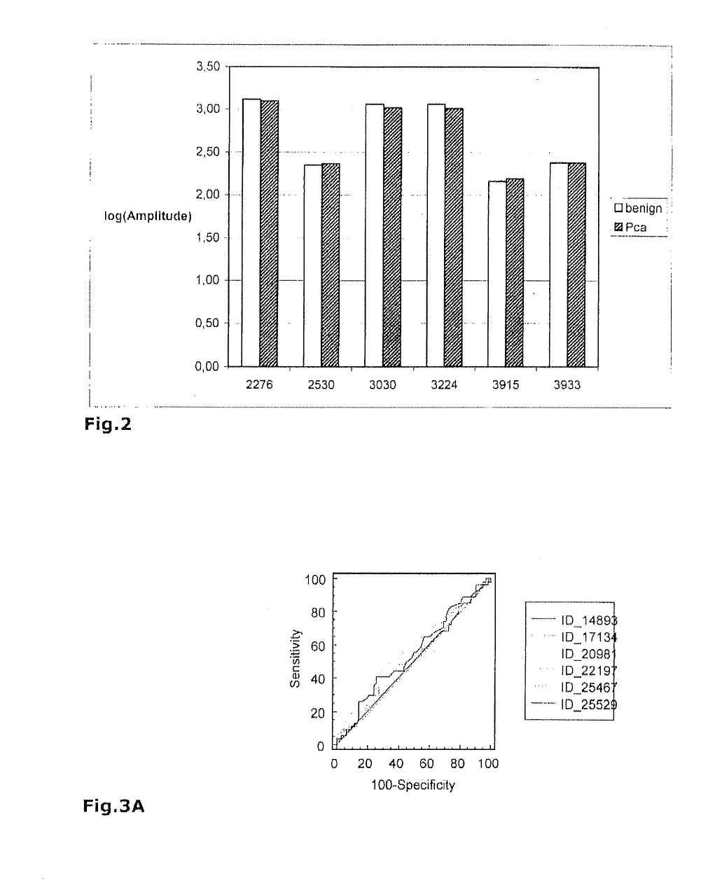

FIG. 2 shows a determination of the amplitudes of the six markers found in this way. It is found that no significant differences occur between prostate carcinoma patients and the control group. Subsequently, the discriminatory value of the biomarkers was examined by means of an ROC (receiver operator characteristic curves) analysis.

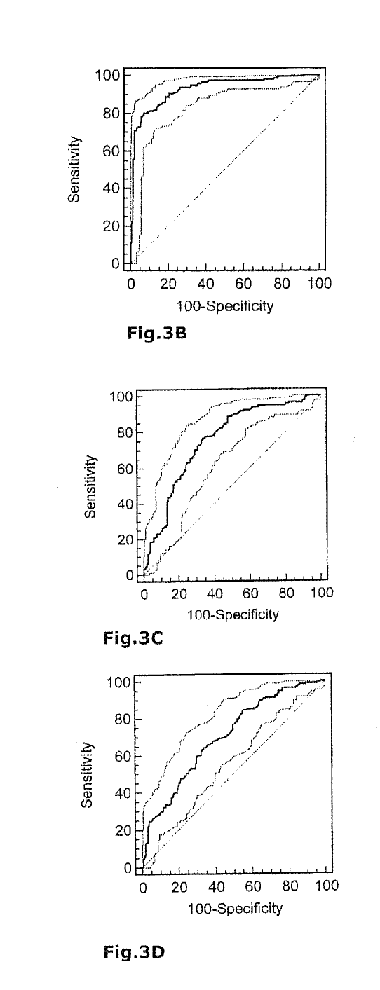

FIG. 3a shows corresponding analyses for six markers that are described in WO 01/25791 and could be found in urine samples.

FIG. 3b shows a corresponding ROC examination of the biomarkers according to the invention with Nos. 142 to 201 of the present patent application. FIG. 3c shows the ROC analysis of a subgroup of the markers according to the invention (12 markers).

FIG. 3d shows that the significance is clearly higher than that of WO 01/25791 even if only three biomarkers according to the invention were used.

1. A method for the diagnosis of prostate diseases, comprising the step of determining an amplitude or the presence or absence of at least one polypeptide marker in a sample, wherein said polypeptide marker is selected from markers 1 to 141, which are characterized by the following values for the molecular masses and migration times:

| Number | Mass (Da) | CE time [min] |
|---|---|---|
| 1 | 858.4 | 23.3 |
| 2 | 911.5 | 25.9 |
| 3 | 1016.3 | 35.7 |
| 4 | 1016.5 | 25.8 |
| 5 | 1050.5 | 26.9 |
| 6 | 1068.6 | 21.7 |
| 7 | 1096.5 | 26.1 |
| 8 | 1128.5 | 25.7 |
| 9 | 1134.6 | 23.6 |
| 10 | 1154.6 | 25.7 |
| 11 | 1157.6 | 37.4 |
| 12 | 1179.6 | 27.1 |
| 13 | 1186.6 | 22.4 |
| 14 | 1191.6 | 36.1 |
| 15 | 1194.6 | 26.7 |
| 16 | 1200.6 | 24.2 |
| 17 | 1216.6 | 24.3 |
| 18 | 1225.6 | 26.3 |
| 19 | 1257.5 | 34.1 |
| 20 | 1265.6 | 27.1 |
| 21 | 1312.7 | 22.4 |
| 22 | 1358.4 | 36.5 |
| 23 | 1392.7 | 21.7 |
| 24 | 1449.7 | 21.8 |
| 25 | 1487.7 | 29.6 |
| 26 | 1523.7 | 22.0 |
| 27 | 1525.5 | 37.2 |
| 28 | 1552.6 | 37.2 |
| 29 | 1576.7 | 26.5 |
| 30 | 1576.7 | 46.0 |
| 31 | 1579.8 | 20.1 |
| 32 | 1584.6 | 37.7 |
| 33 | 1588.8 | 30.2 |
| 34 | 1592.8 | 22.2 |
| 35 | 1600.6 | 37.9 |
| 36 | 1627.8 | 29.5 |
| 37 | 1631.8 | 47.0 |
| 38 | 1634.9 | 29.7 |
| 39 | 1636.8 | 22.5 |
| 40 | 1640.8 | 28.1 |
| 41 | 1649.8 | 22.6 |
| 42 | 1680.8 | 30.0 |
| 43 | 1684.7 | 31.5 |
| 44 | 1687.6 | 37.8 |
| 45 | 1706.9 | 22.7 |
| 46 | 1714.6 | 37.9 |
| 47 | 1725.7 | 38.4 |
| 48 | 1728.8 | 36.8 |
| 19 | 1731.8 | 22.7 |
| 50 | 1755 | 31.4 |
| 51 | 1769.8 | 28.2 |
| 52 | 1783.9 | 39.9 |
| 53 | 1794 | 32.4 |
| 54 | 1806.9 | 23.1 |
| 55 | 1813.8 | 31.7 |
| 56 | 1819.9 | 23.4 |
| 57 | 1825.9 | 20.1 |
| 58 | 1844.6 | 34.2 |
| 59 | 1854.9 | 41.4 |
| 60 | 1860.4 | 33.6 |
| 61 | 1878.7 | 30.9 |
| 62 | 1882.9 | 20.3 |
| 63 | 1911.1 | 25.1 |
| 64 | 1925.9 | 23.3 |
| 65 | 1945.1 | 33.7 |
| 66 | 1950.9 | 35.8 |
| 67 | 1955.9 | 28.1 |
| 68 | 1969.9 | 25.3 |
| 69 | 1993 | 27.1 |
| 70 | 2031 | 32.6 |
| 71 | 2133 | 25.9 |
| 72 | 2157.1 | 22.2 |
| 73 | 2168.9 | 33.9 |
| 74 | 2184.8 | 34.2 |
| 75 | 2188 | 39.9 |
| 76 | 2210.9 | 37.7 |
| 77 | 2282.1 | 34.0 |
| 78 | 2355.2 | 22.7 |
| 19 | 2356.8 | 35.5 |
| 80 | 2414.7 | 35.6 |
| 81 | 2483.2 | 27.7 |
| 82 | 2570.3 | 42.8 |
| 83 | 2577.3 | 24.7 |
| 84 | 2599.3 | 28.0 |
| 85 | 2668.4 | 42.1 |
| 86 | 2682.2 | 22.5 |
| 87 | 2702.1 | 38.2 |
| 88 | 2726.4 | 43.2 |
| 89 | 2742.4 | 42.3 |
| 90 | 2751.5 | 29.2 |
| 91 | 2753.4 | 36.3 |
| 92 | 2754.3 | 29.7 |
| 93 | 2799.2 | 25.1 |
| 94 | 2854.5 | 34.9 |
| 95 | 2977.6 | 29.1 |
| 96 | 3001.5 | 35.5 |
| 97 | 3013.2 | 22.3 |
| 98 | 3021.5 | 23.5 |
| 99 | 3048 | 28.7 |
| 100 | 3092 | 29.7 |
| 101 | 3139.5 | 29.6 |
| 102 | 3145.6 | 38.8 |
| 103 | 3166.4 | 22.1 |
| 104 | 3168.4 | 24.7 |
| 105 | 3193.3 | 22.6 |
| 106 | 3256.6 | 33.1 |
| 107 | 3260.5 | 41.6 |
| 108 | 3292.7 | 39.5 |
| 109 | 3409.7 | 32.2 |
| 110 | 3425.7 | 31.3 |
| 111 | 3426.5 | 27.8 |
| 112 | 3530.6 | 26.2 |
| 113 | 3559.8 | 24.9 |
| 114 | 3657.8 | 40.7 |
| 115 | 3718.8 | 32.5 |
| 116 | 3734.9 | 32.5 |
| 117 | 3765.5 | 20.2 |
| 118 | 3775.7 | 25.9 |
| 119 | 3788.9 | 25.3 |
| 120 | 3858.9 | 25.8 |
| 121 | 3968.7 | 21.1 |
| 122 | 3986.8 | 20.6 |
| 123 | 3996.7 | 20.9 |
| 124 | 4043.7 | 20.4 |
| 125 | 4045 | 26.4 |
| 126 | 4098 | 24.6 |
| 127 | 4218 | 26.1 |
| 128 | 4353 | 20.2 |
| 129 | 4405 | 20.7 |
| 130 | 4410 | 20.0 |
| 131 | 4436.1 | 26.3 |
| 132 | 4672 | 23.3 |
| 133 | 4863.3 | 26.7 |
| 134 | 5000.2 | 24.4 |
| 135 | 6169.9 | 24.6 |
| 136 | 8837.7 | 21.1 |
| 137 | 8854 | 21.1 |
| 138 | 9866.8 | 20.9 |
| 139 | 10342.3 | 23.0 |
| 140 | 10753.7 | 19.7 |
| 141 | 10770.2 | 19.6 |

wherein said sample is a urine sample or seminal fluid sample.

2. The method according to claim 1, wherein an evaluation of the determined presence or absence or amplitude of markers 1 to 141 is effected by using the following reference values:

| | | | Pathological | | Physiological | |
|---|---|---|---|---|---|---|
| | | | prostate gland | | prostate gland | |
| | | CE time | | Mean | | Mean |
| No. | Mass (Da) | (min) | Frequency | amp. | Frequency | amp. |
| 1 | 858.4 | 23.3 | 0.56 | 45 | 0.77 | 123 |
| 2 | 911.5 | 25.9 | 0.73 | 106 | 0.86 | 260 |
| 3 | 1016.3 | 35.7 | 0.96 | 497 | 0.90 | 947 |
| 4 | 1016.5 | 25.8 | 0.95 | 385 | 0.97 | 941 |
| 5 | 1050.5 | 26.9 | 0.75 | 103 | 0.90 | 356 |
| 6 | 1068.6 | 21.7 | 0.54 | 62 | 0.78 | 225 |
| 7 | 1096.5 | 26.1 | 0.89 | 1657 | 0.96 | 3708 |
| 8 | 1128.5 | 25.7 | 0.58 | 60 | 0.80 | 158 |
| 9 | 1134.6 | 23.6 | 0.60 | 67 | 0.80 | 279 |
| 10 | 1154.6 | 25.7 | 0.57 | 95 | 0.80 | 252 |
| 11 | 1157.6 | 37.4 | 0.88 | 457 | 0.89 | 1152 |
| 12 | 1179.6 | 27.1 | 0.78 | 186 | 0.80 | 405 |
| 13 | 1186.6 | 22.4 | 0.76 | 169 | 0.82 | 386 |
| 14 | 1191.6 | 36.1 | 0.55 | 79 | 0.76 | 274 |
| 15 | 1194.6 | 26.7 | 0.94 | 924 | 0.98 | 1575 |
| 16 | 1200.6 | 24.2 | 0.99 | 2465 | 0.99 | 5193 |
| 17 | 1216.6 | 24.3 | 0.88 | 432 | 0.85 | 828 |
| 18 | 1225.6 | 26.3 | 0.87 | 155 | 0.85 | 256 |
| 19 | 1257.5 | 34.1 | 1.00 | 3161 | 0.96 | 2149 |
| 20 | 1265.6 | 27.1 | 0.86 | 2016 | 0.92 | 3539 |
| 21 | 1312.7 | 22.4 | 0.51 | 174 | 0.81 | 729 |
| 22 | 1358.4 | 36.5 | 0.99 | 1848 | 0.95 | 1405 |
| 23 | 1392.7 | 21.7 | 1.00 | 3689 | 0.97 | 2739 |
| 24 | 1449.7 | 21.8 | 1.00 | 4310 | 0.97 | 3020 |
| 25 | 1487.7 | 29.6 | 0.88 | 418 | 0.51 | 234 |
| 26 | 1523.7 | 22.0 | 0.95 | 4303 | 0.96 | 3040 |
| 27 | 1525.5 | 37.2 | 1.00 | 2319 | 0.93 | 1817 |
| 28 | 1552.6 | 37.2 | 0.99 | 2766 | 0.91 | 1862 |
| 29 | 1576.7 | 26.5 | 0.99 | 1736 | 0.93 | 921 |
| 30 | 1576.7 | 46.0 | 0.92 | 2959 | 0.57 | 1199 |
| 31 | 1579.8 | 20.1 | 0.98 | 6547 | 0.98 | 4228 |
| 32 | 1584.6 | 37.7 | 0.87 | 382 | 0.76 | 300 |
| 33 | 1588.8 | 30.2 | 0.94 | 596 | 0.46 | 114 |
| 34 | 1592.8 | 22.2 | 0.83 | 480 | 0.52 | 201 |
| 35 | 1600.6 | 37.9 | 0.88 | 388 | 0.76 | 232 |
| 36 | 1627.8 | 29.5 | 0.81 | 253 | 0.67 | 158 |
| 37 | 1631.8 | 47.0 | 0.96 | 11650 | 0.65 | 4248 |
| 38 | 1634.9 | 29.7 | 0.85 | 522 | 0.67 | 272 |
| 39 | 1636.8 | 22.5 | 1.00 | 10631 | 0.98 | 7771 |
| 40 | 1640.8 | 28.1 | 0.84 | 162 | 0.41 | 50 |
| 41 | 1649.8 | 22.6 | 0.93 | 699 | 0.82 | 422 |
| 42 | 1680.8 | 30.0 | 0.89 | 2045 | 0.86 | 1483 |
| 43 | 1684.7 | 31.5 | 0.99 | 2814 | 0.98 | 1441 |
| 44 | 1687.6 | 37.8 | 0.93 | 217 | 0.80 | 154 |
| 45 | 1706.9 | 22.7 | 1.00 | 796 | 0.93 | 408 |
| 46 | 1714.6 | 37.9 | 0.71 | 116 | 0.45 | 48 |
| 47 | 1725.7 | 38.4 | 1.00 | 2078 | 0.90 | 1407 |
| 48 | 1728.8 | 36.8 | 0.79 | 221 | 0.67 | 124 |
| 49 | 1731.8 | 22.7 | 0.88 | 172 | 0.69 | 108 |
| 50 | 1755 | 31.4 | 1.00 | 9478 | 0.92 | 3966 |
| 51 | 1769.8 | 28.2 | 0.93 | 403 | 0.69 | 137 |
| 52 | 1783.9 | 39.9 | 0.76 | 240 | 0.40 | 112 |
| 53 | 1794 | 32.4 | 0.88 | 287 | 0.78 | 202 |
| 54 | 1806.9 | 23.1 | 0.83 | 188 | 0.60 | 112 |
| 55 | 1813.8 | 31.7 | 0.99 | 2221 | 0.95 | 983 |
| 56 | 1819.9 | 23.4 | 1.00 | 3799 | 0.93 | 2664 |
| 57 | 1825.9 | 20.1 | 0.95 | 1494 | 0.90 | 926 |
| 58 | 1844.6 | 34.2 | 0.88 | 326 | 0.54 | 137 |
| 59 | 1854.9 | 41.4 | 0.95 | 7712 | 0.84 | 4491 |
| 60 | 1860.4 | 33.6 | 0.85 | 401 | 0.64 | 194 |
| 61 | 1878.7 | 30.9 | 0.84 | 963 | 0.69 | 534 |
| 62 | 1882.9 | 20.3 | 1.00 | 18028 | 0.98 | 12004 |
| 63 | 1911.1 | 25.1 | 0.99 | 62532 | 0.96 | 39024 |
| 64 | 1925.9 | 23.3 | 0.75 | 120 | 0.28 | 44 |
| 65 | 1945.1 | 33.7 | 0.86 | 197 | 0.74 | 180 |
| 66 | 1950.9 | 35.8 | 0.97 | 593 | 0.77 | 357 |
| 67 | 1955.9 | 28.1 | 0.82 | 345 | 0.44 | 86 |
| 68 | 1969.9 | 25.3 | 0.89 | 915 | 0.61 | 242 |
| 69 | 1993 | 27.1 | 0.80 | 104 | 0.34 | 33 |
| 70 | 2031 | 32.6 | 0.92 | 838 | 0.80 | 538 |
| 71 | 2133 | 25.9 | 0.98 | 714 | 0.70 | 278 |
| 72 | 2157.1 | 22.2 | 0.79 | 347 | 0.90 | 736 |
| 73 | 2168.9 | 33.9 | 0.99 | 706 | 0.91 | 493 |
| 74 | 2184.8 | 34.2 | 0.95 | 610 | 0.88 | 300 |
| 75 | 2188 | 39.9 | 0.97 | 1452 | 0.81 | 606 |
| 76 | 2210.9 | 37.7 | 0.84 | 274 | 0.64 | 183 |
| 77 | 2282.1 | 34.0 | 0.99 | 5409 | 0.99 | 5449 |
| 78 | 2355.2 | 22.7 | 0.99 | 554 | 0.97 | 956 |
| 79 | 2356.8 | 35.5 | 0.98 | 571 | 0.75 | 230 |
| 80 | 2414.7 | 35.6 | 0.78 | 471 | 0.61 | 164 |
| 81 | 2483.2 | 27.7 | 0.76 | 155 | 0.84 | 353 |
| 82 | 2570.3 | 42.8 | 0.99 | 5993 | 0.80 | 3366 |
| 83 | 2577.3 | 24.7 | 0.96 | 297 | 0.82 | 516 |
| 84 | 2599.3 | 28.0 | 0.95 | 1856 | 0.79 | 180 |
| 85 | 2668.4 | 42.1 | 0.90 | 529 | 0.72 | 333 |
| 86 | 2682.2 | 22.5 | 0.99 | 844 | 0.91 | 588 |
| 87 | 2702.1 | 38.2 | 0.82 | 332 | 0.64 | 137 |
| 88 | 2726.4 | 43.2 | 0.99 | 4394 | 0.80 | 2280 |
| 89 | 2742.4 | 42.3 | 0.87 | 592 | 0.50 | 188 |
| 90 | 2751.5 | 29.2 | 0.87 | 239 | 0.55 | 139 |
| 91 | 2753.4 | 36.3 | 0.86 | 150 | 0.64 | 79 |
| 92 | 2754.3 | 29.7 | 0.88 | 318 | 0.77 | 182 |
| 93 | 2799.2 | 25.1 | 1.00 | 3236 | 1.00 | 2169 |
| 94 | 2854.5 | 34.9 | 0.99 | 3786 | 0.97 | 2215 |
| 95 | 2977.6 | 29.1 | 0.84 | 179 | 0.47 | 66 |
| 96 | 3001.5 | 35.5 | 0.99 | 12587 | 0.98 | 7515 |
| 97 | 3013.2 | 22.3 | 0.91 | 1824 | 0.98 | 3609 |
| 98 | 3021.5 | 23.5 | 1.00 | 1642 | 0.85 | 1055 |
| 99 | 3048 | 28.7 | 0.82 | 230 | 0.72 | 112 |
| 100 | 3092 | 29.7 | 0.99 | 780 | 0.89 | 618 |
| 101 | 3139.5 | 29.6 | 0.97 | 1549 | 0.86 | 673 |
| 102 | 3145.6 | 38.8 | 0.97 | 1147 | 0.84 | 663 |
| 103 | 3166.4 | 22.1 | 0.82 | 409 | 0.51 | 159 |
| 104 | 3168.4 | 24.7 | 0.82 | 211 | 0.91 | 477 |
| 105 | 3193.3 | 22.6 | 0.81 | 383 | 0.88 | 783 |
| 106 | 3256.6 | 33.1 | 0.90 | 1008 | 0.71 | 564 |
| 107 | 3260.5 | 41.6 | 0.86 | 371 | 0.55 | 205 |
| 108 | 3292.7 | 39.5 | 0.99 | 5967 | 0.91 | 3920 |
| 109 | 3409.7 | 32.2 | 0.89 | 386 | 0.84 | 287 |
| 110 | 3425.7 | 31.3 | 1.00 | 1098 | 0.97 | 1200 |
| 111 | 3426.5 | 27.8 | 0.81 | 136 | 0.45 | 45 |
| 112 | 3530.6 | 26.2 | 0.95 | 775 | 0.73 | 317 |
| 113 | 3559.8 | 24.9 | 0.96 | 896 | 0.80 | 633 |
| 114 | 3657.8 | 40.7 | 0.96 | 964 | 0.80 | 648 |
| 115 | 3718.8 | 32.5 | 0.97 | 364 | 0.95 | 695 |
| 116 | 3734.9 | 32.5 | 0.91 | 200 | 0.93 | 410 |
| 117 | 3765.5 | 20.2 | 0.60 | 199 | 0.26 | 40 |
| 118 | 3775.7 | 25.9 | 0.99 | 1537 | 0.88 | 1110 |
| 119 | 3788.9 | 25.3 | 0.78 | 258 | 0.57 | 144 |
| 120 | 3858.9 | 25.8 | 0.97 | 975 | 0.82 | 690 |
| 121 | 3968.7 | 21.1 | 0.95 | 1430 | 0.83 | 904 |
| 122 | 3986.8 | 20.6 | 0.99 | 4017 | 0.86 | 1722 |
| 123 | 3996.7 | 20.9 | 0.83 | 707 | 0.39 | 197 |
| 124 | 4043.7 | 20.4 | 0.92 | 1038 | 0.67 | 496 |
| 125 | 4045 | 26.4 | 0.98 | 2449 | 0.88 | 1561 |
| 126 | 4098 | 24.6 | 0.94 | 370 | 0.90 | 780 |
| 127 | 4218 | 26.1 | 0.97 | 4520 | 0.88 | 2787 |
| 128 | 4353 | 20.2 | 0.98 | 4815 | 0.88 | 2186 |
| 129 | 4405 | 20.7 | 0.86 | 704 | 0.59 | 244 |
| 130 | 4410 | 20.0 | 0.82 | 997 | 0.60 | 472 |
| 131 | 4436.1 | 26.3 | 0.95 | 2663 | 0.87 | 1538 |
| 132 | 4672 | 23.3 | 0.90 | 309 | 0.77 | 196 |
| 133 | 4863.3 | 26.7 | 0.61 | 158 | 0.76 | 389 |
| 134 | 5000.2 | 24.4 | 0.99 | 2538 | 0.98 | 1743 |
| 135 | 6169.9 | 24.6 | 0.78 | 444 | 0.46 | 282 |
| 136 | 8837.7 | 21.1 | 0.94 | 1669 | 0.71 | 1430 |
| 137 | 8854 | 21.1 | 0.63 | 441 | 0.86 | 1383 |
| 138 | 9866.8 | 20.9 | 0.99 | 1874 | 0.83 | 944 |
| 139 | 10342.3 | 23.0 | 0.77 | 1771 | 0.35 | 279 |
| 140 | 10753.7 | 19.7 | 0.76 | 4585 | 0.33 | 247 |
| 141 | 10770.2 | 19.6 | 0.52 | 2190 | 0.14 | 59 |

3. The method according to claim 1, wherein at least three or at least five or at least ten polypeptide markers as defined in claim 1 are used.

4. A method for the differential diagnosis between prostate cancer (PCA) and benign prostate hyperplasia (BPH), comprising the step of determining the presence or absence of at least one polypeptide marker in a sample, wherein said polypeptide marker is selected from markers 142 to 201, which are characterized by the following values for the molecular masses and migration times:

| | | CE time |
|---|---|---|
| No. | Mass (Da) | (min) |
| 142 | 1210.4 | 36.5 |
| 143 | 1210.6 | 20.9 |
| 144 | 1234.6 | 27.4 |
| 145 | 1235.6 | 26.7 |
| 146 | 1268.6 | 21.4 |
| 147 | 1276.5 | 36.0 |
| 148 | 1390.5 | 37.1 |
| 149 | 1440.6 | 24.3 |
| 150 | 1467.7 | 21.7 |
| 151 | 1491.8 | 39.9 |
| 152 | 1495.6 | 37.4 |
| 153 | 1510.7 | 24.3 |
| 154 | 1523.8 | 40.5 |
| 155 | 1552.6 | 37.2 |
| 156 | 1579.8 | 29.8 |
| 157 | 1584.6 | 37.7 |
| 158 | 1624.6 | 37.7 |
| 159 | 1664.8 | 29.8 |
| 160 | 1680.8 | 30.0 |
| 161 | 1687.6 | 37.8 |
| 162 | 1706.9 | 22.7 |
| 163 | 1714.6 | 37.9 |
| 164 | 1725.7 | 38.4 |
| 165 | 1783.9 | 39.9 |
| 166 | 1794.9 | 24.0 |
| 167 | 1822.8 | 30.9 |
| 168 | 1825.9 | 20.1 |
| 169 | 1864.0 | 44.1 |
| 170 | 1882.9 | 20.3 |
| 171 | 1925.9 | 23.3 |
| 172 | 1964.0 | 31.8 |
| 173 | 2089.0 | 23.7 |
| 174 | 2118.0 | 33.0 |
| 175 | 2133.0 | 25.9 |
| 176 | 2170.0 | 33.4 |
| 177 | 2189.1 | 26.8 |
| 178 | 2282.1 | 34.0 |
| 179 | 2298.1 | 33.9 |
| 180 | 2442.2 | 34.1 |
| 181 | 2485.2 | 34.4 |
| 182 | 2599.3 | 28.0 |
| 183 | 2686.9 | 29.1 |
| 184 | 2695.3 | 23.5 |
| 185 | 2839.0 | 24.2 |
| 186 | 3092.0 | 29.7 |
| 187 | 3121.4 | 30.3 |
| 188 | 3248.5 | 30.7 |
| 189 | 3302.8 | 23.2 |
| 190 | 3333.8 | 23.8 |
| 191 | 3409.7 | 32.2 |
| 192 | 3425.7 | 31.3 |
| 193 | 3457.7 | 31.5 |
| 194 | 3478.4 | 41.8 |
| 195 | 3524.6 | 32.4 |
| 196 | 3589.8 | 25.1 |
| 197 | 3765.5 | 20.2 |
| 198 | 3839.9 | 19.7 |
| 199 | 4290.0 | 28.8 |
| 200 | 6650.9 | 25.6 |
| 201 | 10770.2 | 19.6 |

wherein said sample is a urine sample or seminal fluid sample.

5. The method according to claim 4, wherein an evaluation of the determined amplitude, presence or absence is effected by using the following reference values:

| | | | PCa | | BPH | |
|---|---|---|---|---|---|---|
| | Mass | CE time | | Mean | Frequency | Mean |
| No. | (Da) | (min) | Frequency (%) | amp. | (%) | amp. |
| 142 | 1210.4 | 36.5 | 0.50 | 149 | 0.61 | 243 |
| 143 | 1210.6 | 20.9 | 0.65 | 121 | 0.54 | 101 |
| 144 | 1234.6 | 27.4 | 0.96 | 841 | 0.98 | 977 |
| 145 | 1235.6 | 26.7 | 0.69 | 550 | 0.55 | 458 |
| 146 | 1268.6 | 21.4 | 0.37 | 52 | 0.46 | 86 |
| 147 | 1276.5 | 36.0 | 0.98 | 2879 | 0.99 | 3241 |
| 148 | 1390.5 | 37.1 | 1.00 | 19627 | 0.97 | 21822 |
| 149 | 1440.6 | 24.3 | 0.66 | 135 | 0.62 | 107 |
| 150 | 1467.7 | 21.7 | 0.41 | 89 | 0.54 | 102 |
| 151 | 1491.8 | 39.9 | 0.68 | 217 | 0.76 | 269 |
| 152 | 1495.6 | 37.4 | 0.68 | 134 | 0.79 | 162 |
| 153 | 1510.7 | 24.3 | 0.39 | 47 | 0.50 | 77 |
| 154 | 1523.8 | 40.5 | 0.98 | 7023 | 0.98 | 7856 |
| 155 | 1552.6 | 37.2 | 0.98 | 2130 | 0.99 | 2530 |
| 156 | 1579.8 | 29.8 | 0.98 | 1824 | 0.99 | 2061 |
| 157 | 1584.6 | 37.7 | 0.82 | 301 | 0.89 | 397 |
| 158 | 1624.6 | 37.7 | 0.99 | 1020 | 0.99 | 1159 |
| 159 | 1664.8 | 29.8 | 0.92 | 386 | 0.94 | 475 |
| 160 | 1680.8 | 30.0 | 0.82 | 1626 | 0.92 | 2182 |
| 161 | 1687.6 | 37.8 | 0.88 | 161 | 0.88 | 193 |
| 162 | 1706.9 | 22.7 | 0.99 | 705 | 0.97 | 624 |
| 163 | 1714.6 | 37.9 | 0.52 | 72 | 0.67 | 105 |
| 164 | 1725.7 | 38.4 | 1.00 | 1646 | 0.99 | 1964 |
| 165 | 1783.9 | 39.9 | 0.63 | 175 | 0.73 | 224 |
| 166 | 1794.9 | 24.0 | 0.98 | 1179 | 0.96 | 1305 |
| 167 | 1822.8 | 30.9 | 0.67 | 192 | 0.59 | 148 |
| 168 | 1825.9 | 20.1 | 0.95 | 1629 | 0.96 | 1370 |
| 169 | 1864.0 | 44.1 | 0.70 | 874 | 0.77 | 1182 |
| 170 | 1882.9 | 20.3 | 0.99 | 18958 | 0.99 | 17650 |
| 171 | 1925.9 | 23.3 | 0.74 | 121 | 0.62 | 105 |
| 172 | 1964.0 | 31.8 | 0.54 | 96 | 0.42 | 71 |
| 173 | 2089.0 | 23.7 | 0.56 | 76 | 0.46 | 52 |
| 174 | 2118.0 | 33.0 | 0.43 | 42 | 0.55 | 61 |
| 175 | 2133.0 | 25.9 | 0.98 | 711 | 0.94 | 589 |
| 176 | 2170.0 | 33.4 | 0.34 | 201 | 0.48 | 295 |
| 177 | 2189.1 | 26.8 | 0.94 | 889 | 0.92 | 640 |
| 178 | 2282.1 | 34.0 | 1.00 | 5015 | 0.99 | 5720 |
| 179 | 2298.1 | 33.9 | 0.49 | 95 | 0.58 | 127 |
| 180 | 2442.2 | 34.1 | 0.97 | 553 | 0.98 | 708 |
| 181 | 2485.2 | 34.4 | 0.49 | 42 | 0.57 | 67 |
| 182 | 2599.3 | 28.0 | 0.94 | 1303 | 0.93 | 1675 |
| 183 | 2686.9 | 29.1 | 0.54 | 102 | 0.66 | 117 |
| 184 | 2695.3 | 23.5 | 0.99 | 2295 | 1.00 | 2058 |
| 185 | 2839.0 | 24.2 | 0.77 | 589 | 0.86 | 687 |
| 186 | 3092.0 | 29.7 | 0.95 | 704 | 0.99 | 854 |
| 187 | 3121.4 | 30.3 | 0.93 | 585 | 0.94 | 690 |
| 188 | 3248.5 | 30.7 | 1.00 | 1184 | 1.00 | 1363 |
| 189 | 3302.8 | 23.2 | 0.65 | 102 | 0.68 | 135 |
| 190 | 3333.8 | 23.8 | 0.41 | 128 | 0.32 | 77 |
| 191 | 3409.7 | 32.2 | 0.81 | 357 | 0.92 | 508 |
| 192 | 3425.7 | 31.3 | 1.00 | 1132 | 1.00 | 1339 |
| 193 | 3457.7 | 31.5 | 1.00 | 13651 | 1.00 | 15121 |
| 194 | 3478.4 | 41.8 | 0.53 | 315 | 0.64 | 368 |
| 195 | 3524.6 | 32.4 | 0.49 | 285 | 0.61 | 397 |
| 196 | 3589.8 | 25.1 | 0.93 | 778 | 0.91 | 699 |
| 197 | 3765.5 | 20.2 | 0.64 | 200 | 0.43 | 132 |
| 198 | 3839.9 | 19.7 | 0.92 | 1868 | 0.92 | 1621 |
| 199 | 4290.0 | 28.8 | 0.97 | 2383 | 0.99 | 2698 |
| 200 | 6650.9 | 25.6 | 0.69 | 161 | 0.54 | 134 |
| 201 | 10770.2 | 19.6 | 0.44 | 1814 | 0.60 | 2746 |

6. The method according to claim 4, wherein at least three or at least four or at least five or at least ten or all polypeptide markers as defined in claim 4 are used.

7. The method according to claim 1, wherein capillary electrophoresis, HPLC, gas-phase ion spectrometry and/or mass spectrometry is used for determining the amplitude or detecting the presence or absence of the polypeptide marker or markers.

8. The method according claim 1, wherein a capillary electrophoresis is performed before the molecular mass of the polypeptide markers is measured.

9. The method according claim 1, wherein mass spectrometry is used for detecting the presence or absence or for determining the amplitude of the polypeptide marker or markers.

10. The method according to claim 1, wherein a separation of proteins of >20 kDa is effected before the measurement.

11-12. (canceled)
